(19) United States
(12) Patent Application Publication
(10) Pub. No.: US 2024/0000390 A1
(43) Pub. Date: Jan. 4, 2024

(54) MEASUREMENT-CONTACT OPTIMIZATION FOR VOLTAGE MEASUREMENT SYSTEMS

(71) Applicant: Siemens Healthcare GmbH, Erlangen (DE)

(72) Inventor: Ulrich BATZER, Spardorf (DE)

(73) Assignee: Siemens Healthcare GmbH, Erlangen (DE)

(21) Appl. No.: 18/342,864

(22) Filed: Jun. 28, 2023

(30) Foreign Application Priority Data
Jun. 30, 2022 (DE) 10 2022 206 709.6

(51) Int. Cl. A61B 5/00; A61B 5/053

(52) U.S. Cl. CPC A61B 5/70; A61B 5/6892; A61B 5/7225; A61B 5/053; A61B 2562/0247

(57) ABSTRACT

One or more example embodiments relates to a measurement system for measuring bio-electric signals from a patient, comprising a sensor electrode; and a mechanical mounting for the sensor electrode, the mechanical mounting being compressible at least partially by a weight of the patient, the mechanical mounting including a frame structure and a compressible supporting structure, wherein the mechanical mounting is attachable to a substrate of the measurement system to support the sensor electrode against the substrate, the compressible supporting structure is beneath the sensor electrode, the frame structure at least partially surrounds the supporting structure, and in an unloaded state, the supporting structure protrudes beyond the frame structure.

### CROSS-REFERENCE TO RELATED APPLICATION(S)

The present application claims priority under 35 U.S.C. § 119 to German Patent Application No. 10 2022 206 709.6, filed Jun. 30, 2022, the entire contents of which are incorporated herein by reference.

### FIELD

One or more example embodiments relates to a measurement system for measuring bio-electric signals from a patient, which is designed to acquire a patient positioning relative to the measurement system, and relates to a corresponding signal measurement circuit, to a corresponding differential voltage measurement system, and to a corresponding method.

### RELATED ART

Voltage measurement systems, in particular differential voltage measurement systems, for measuring bio-electric signals are used for example in medicine to measure electrocardiograms (ECG), electroencephalograms (EEG) or electromyograms (EMG).

Measuring heart activity using the voltage measurement systems mentioned is necessary in particular for imaging the heart, in order to adapt the imaging process to the strongly pronounced movement of the heart during the heartbeat. Conventional sensors, which must be attached to the patient's body, are used for this purpose. One possible way of measuring the heartbeat is a capacitive ECG, in which an ECG signal is picked up purely capacitively without the patient having direct contact with the sensor, in particular through the patient's clothing. The signal amplitude must preferably be large in order to achieve a good signal quality of the heartbeat signal. This can be achieved by a large capacitance between patient and sensor. The capacitance can be influenced via the size of the coupling area between sensor and patient. The larger the coupling area, the larger the capacitance achieved.

It is particularly comfortable for the patient if the capacitive ECG measurement can be performed without putting on or attaching individual sensors. It is known for this purpose to integrate the sensor equipment into the surface of a patient couch, for example of an imaging facility, or into an underlay mat or into a (seat) backrest, so that the voltage measurement can take place as soon as the patient is in position on the patient couch or the seat. German patent DE 10 2015 218 298 B3, for example, describes such an apparatus.

For a smooth examination procedure, it is important to identify quickly whether, in terms of acquiring a bio-electric signal, the patient is correctly positioned or lying correctly on, for instance, the underlay mat, in order to be able to give immediate feedback and, if necessary, change the placement of the patient.

### SUMMARY

One or more example embodiments provides alternative means to acquire reliably and quickly the quality of a present patient position or placement, and hence to acquire a measurement signal quality expected with the present patient position. In particular, a means for repositioning the patient or placing the patient in a new position, based on the acquired quality or position quality is provided.

This object is achieved by a measurement system for measuring bio-electric measurement signals from a patient, a signal measurement circuit for a differential voltage measurement system for measuring bio-electric measurement signals from a patient, a differential voltage measurement system, and a corresponding method according to the independent claims. Preferred and/or alternative advantageous configurations are the subject matter of the dependent claims and the following description, wherein individual features of different exemplary embodiments or variants can also be combined to create new exemplary embodiments or variants.

### BRIEF DESCRIPTION OF THE DRAWINGS

The following description of the exemplary embodiments, which are explained in greater detail in conjunction with the drawings, will clarify and elucidate the above-described properties, features and advantages of this invention, and the manner in which they are achieved. This description does not restrict the invention to these exemplary embodiments. Identical components are denoted by the same reference signs in the different figures, which are generally not shown to scale and in which:

FIG. 1 shows a view of a measurement system according to one or more example embodiments;

FIG. 2 shows a view of a differential voltage measurement system according to one or more example embodiments;

FIG. 3 shows a view of a differential voltage measurement system comprising two signal measurement circuits according to one or more example embodiments; and

FIG. 4 shows a schematic diagram of a method according to one or more example embodiments.

### DETAILED DESCRIPTION

German patent application DE 10 2016 112 391 A1 proposes impinging, in addition to sensor electrodes, an injection signal in order to be able to assess the signal quality of the measurement signal acquired by the sensor electrodes. The quality of a present patient position can be concluded from the assessed signal quality, and the patient can be repositioned if necessary. At least one separate injection electrode is needed in order to be able to impinge the injection signal, which involves greater material expense and may restrict patient comfort. Furthermore, the coupling of the injection electrodes themselves is unknown, and therefore it is critical with regard to patient protection to assess the feeding-in of the injection signal. Moreover, the injection signal can interfere with further medical devices present during the examination, which can likewise mean a health risk to the patient.

In addition, German patent application DE 10 2021 202 347 A1 describes a measurement system for acquiring bio-electric signals which is designed to set a constant contact pressure independently of a present patient position or his placement relative to the measurement system.

Example embodiments are described with reference to the claimed apparatuses. It may be the case here that features, advantages or alternative embodiment(s) forms which can be directed at a method, for example, are also described or claimed in connection with one of the apparatuses. Corresponding functional features are embodied in this case by corresponding physical modules or units of the apparatuses.

One or more example embodiments relates to a measurement system for measuring bio-electric measurement signals from a patient.

The patient is typically a human being. In principle, the patient may also be an animal.

The measurement system basically has the same construction as the measurement system described in German patent application DE 10 2021 202 347 A1, the entire contents of which are herein incorporated by reference.

The measurement system comprises a sensor electrode. The measurement system also comprises a mechanical mounting for the sensor electrode, which mounting is compressible at least partially and comprises a frame structure and a supporting structure. The compression is typically brought about by a force component applied or introduced by a body of a patient. In particular, the mechanical mounting can be compressed by a weight of the patient. This means that the patient can be positioned in particular above or on the measurement system, more specifically the sensor electrode. The mechanical mounting can be attached to a substrate of the measurement system, and is designed to support the sensor electrode against the substrate. The supporting structure is arranged beneath the sensor electrode, and the frame structure at least partially surrounds the supporting structure. In embodiments, at least the supporting structure of the mechanical mounting is compressible, in particular is designed to be compressible by the weight of the patient. The frame structure, on the other hand, in embodiments is incompressible or significantly harder than the supporting structure. The supporting structure is designed to be higher than the frame structure in the unloaded state. In other words, in the unloaded state, the supporting structure protrudes beyond the frame structure.

In addition, the compressible supporting structure of the mechanical mounting is designed to be conductive, which means that it comprises a conductive material or consists of a conductive material. In particular, the supporting structure is designed to be conductive throughout, i.e. over its entire volume. The supporting structure is covered in the direction of the sensor electrode and in the direction of the substrate by a first upper conductive layer and a first lower conductive layer respectively. This first upper conductive layer and the first lower conductive layer serve to pick up a voltage dropped across the compressible supporting structure. In other words, a predefined current can be impinged onto the supporting structure via the two first conductive layers. In an alternative embodiment, the supporting structure has multiple layers and comprises the first upper conductive layer and the first lower conductive layer and also a conductive compressible material therebetween. The first conductive layers function, according to one or more example embodiments, as measurement electrodes, which lie against the supporting structure. They are constituent parts of a voltage measurement apparatus according to one or more example embodiments, which, on the impinging of a defined current, acquire a voltage dropped across the supporting structure.

In preferred embodiments of the invention, the sensor electrode forms part of a signal measurement circuit of a differential voltage measurement system.

Accordingly, in a further aspect, one or more example embodiments relates to a signal measurement circuit for a differential voltage measurement system for measuring bio-electric signals from a patient. The signal measurement circuit comprises:
a measurement system according to one or more example embodiments;
a measurement amplifier circuit; and
a sensor line between the measurement amplifier circuit and the sensor electrode.

In embodiments of the invention, the sensor electrode is in the form of a flat electrode, preferably a square or rectangular or round flat electrode, and has a film-like structure.

In embodiments of the invention, the sensor line is used to transmit the bio-electric measurement signals acquired by the sensor electrode to the measurement amplifier circuit.

The measurement amplifier circuit preferably comprises an operational amplifier, which can be embodied as what is known as a voltage follower. This means that the negative input of the operational amplifier, also known as the inverting input, is coupled to the output of the operational amplifier, thereby producing a high virtual input impedance at the positive input.

The mechanical mounting according to one or more example embodiments of the sensor electrode is designed to be compressible at least partially, i.e., under a mechanical load, in particular caused by a patient leaning against the measurement system or positioned thereon, the mechanical mounting advantageously changes its shape, in particular its height, at least in the compressible regions or parts. The mechanical mounting joins a substrate of the measurement system, for example a lower outer surface of the measurement system, to the sensor electrode. The mechanical mounting therefore carries and supports the sensor electrode under no pressure/load at a predefined distance from the substrate. The mounting can be attached, for instance welded or adhesively bonded, to the substrate for the purpose of stabilizing the measurement system.

In order to minimize pressure variations at the sensor electrode induced by patient movement, and capacitance changes and tribological effects induced thereby, the mechanical mounting comprises a frame structure and a supporting structure. The supporting structure is arranged beneath the sensor electrode. Thus the supporting structure has at least substantially the same base area as the sensor electrode. If the measurement system is integrated in a mat-like structure, or if size or space specifications do not have to be considered, the sensor electrode and supporting structure preferably have a square base area between 3 cm×3 cm and 7 cm×7 cm. Particularly preferably, the supporting structure and sensor electrode each have a base area of 4 cm×4 cm. In other embodiments, the base area is correspondingly more compact. In this case, the sensor electrode and the supporting structure preferably have a base area of 1 cm×1 cm to a maximum of 2 cm×5 cm. The sensor electrode preferably lies over the entire surface of the supporting structure. The frame structure at least partially surrounds the supporting structure. In preferred embodiments, the frame structure surrounds the supporting structure at least on two opposite sides. In other preferred embodiments, the frame structure completely surrounds the supporting structure. Thus in this case the frame structure is a closed structure around the supporting structure. In embodiments of the measurement system, for a mat-like structure, the frame structure preferably has a width/thickness of 1 cm in a direction perpendicular to the supporting structure. Overall, the dimensions of the base area of a measurement system according to one or more example embodiments can preferably lie between 3 cm (in a spatial direction excluding frame structure) or 5 cm and 9 cm. In embodiments of the measurement system, the frame structure preferably has a thickness/width of 5 mm to 15 mm.

In preferred embodiments of the invention, the compressible conductive supporting structure is formed from a compressible foamed material, preferably from a viscoelastic foamed material, i.e. from a reversibly deformable foamed material. In this case, a polyethylene terephthalate (PET) foamed material or a polyurethane (PU) foamed material can be used, for example.

In further preferred embodiments of the invention, the supporting structure has a lower hardness than the frame structure. This can be achieved by forming the frame structure from a harder foamed material, from plastic, from wood, or another incompressible material. The material of the frame structure is preferably designed to be incompressible or only slightly compressible. For greater patient comfort, in the case of a measurement system for an ECG mat, the frame structure also consists of a slightly compressible foamed material. The frame structure can also be made from a polyethylene plastic.

The measurement system thereby ensures that the supporting structure can be compressed only to the height of the frame structure but barely therebeyond. When the supporting structure has been compressed to the same height/thickness as the frame structure, a force component of the currently acting compression force is introduced into the frame structure. This force component, however, does not cause any further compression of the supporting structure. The compression force component acting on the supporting structure is consequently limited to a defined value, which is predetermined by the frame structure and the height difference between frame structure and supporting structure.

In further preferred embodiments, the compressible conductive supporting structure comprises a conductive foamed material. This is achieved according to one or more example embodiments preferably by a carbon admixture to the foam material. In embodiments of the invention, the carbon component lies between 15 and 50 percent by mass or volume, for example 30, 35, 40, 45 percent by volume. For example, the carbon can be admixed in the form of carbon powder to the foam material before the foamed material is produced.

In a further embodiment of the invention, the first upper conductive layer and the first lower conductive layer comprise a conductive plastic. In a preferred embodiment, the first upper layer and the first lower layer are designed to be incompressible, or less compressible than the supporting structure. PU or PET plastics can again be used as the plastics, which are not foamed for the first conductive layers. The conductivity is also achieved in the first conductive layers via a carbon admixture, in particular a carbon admixture of 15 to 50 percent by mass or volume, again in this case, for example, 30, 35, 40, 45 percent by volume.

The first conductive layers are made particularly preferably from a highly conductive plastic. Particularly preferably, the first conductive layers have a surface resistance of less than 10 kilohms, in particular less than 1 kilohm.

Empirical studies have been used to specify the thickness, or material thickness, of the first conductive layers in a range of 20 μm to 50 μm. The conductive plastic is preferably deployed with a thickness between 20 μm and 30 μm, for example 22 μm or 25 μm.

In embodiments of the invention, the first conductive layers are welded to the supporting structure so that the resistance at the interfaces between conductive layer and supporting structure is also as low as possible.

As described in the introduction, the supporting structure changes its height, or its dimension in at least one spatial direction, as a result of an introduced force component. The ohmic resistance of the supporting structure also changes as a result of compression of the foamed material of the supporting structure. The inventors have discovered that if the force-resistance characteristic curve or the compression-resistance characteristic curve of the supporting structure is known, the currently acting compression force or the compression pressure can be determined. These properties and these characteristic curves can be ascertained experimentally in advance, and are specific to the selected material and the selected geometry of the supporting structure. Via the current impinged via the first upper conductive layer and first lower conductive layer, and the associated picked-up voltage, the resistance can be derived first, and hence the acting compression force can be determined.

A further preferred embodiment of the invention accordingly provides a supporting structure that, in the unloaded state, has an ohmic resistance in the range between 100 kilohms and 1000 kilohms. The ohmic resistance decreases in the compressed state, and decreases further with increasing compression. In other words, in the unloaded state, the ohmic resistance is a maximum and lies in the range stated above. A compression-resistance characteristic curve hence runs between zero and a value in the range stated above.

In embodiments, the mechanical mounting of the measurement system further comprises a carrier structure, which can be compressed by the weight of the patient and which runs beneath the compressible conductive supporting structure and the frame structure. The carrier structure also is designed to be conductive, and is covered in the direction of the sensor electrode and in the direction of the substrate by a second upper conductive layer and a second lower conductive layer respectively for acquiring a voltage dropped across the carrier structure.

The carrier structure is preferably in the form of layers and in particular covers the entire surface, i.e. it has a base area that equals at least the base area of the measurement system. In embodiments, the base area of the carrier structure can also be larger. In embodiments, the carrier structure joins the substrate of the measurement system to the frame structure and/or the supporting structure. The carrier structure is preferably joined, preferably welded or adhesively bonded, both to the substrate and to the frame structure and/or supporting structure.

In embodiments, it is provided that the supporting structure has a lower hardness than the carrier structure. The carrier structure is therefore preferably likewise designed to be compressible and conductive, and preferably is likewise formed from a carbon-enriched foamed material. The carrier structure serves as a flexible mount for the supporting structure and/or the frame structure. The carrier structure is preferably made from a foamed material that is 20% to 30% harder than the foamed material of the supporting structure. In embodiments, the carrier structure serves, in addition to the often incompressible frame structure, to absorb the excess force component applied to the measurement system once the supporting structure has already been compressed to the height of the frame structure. Depending on the material selected for the carrier structure, the carrier structure can have a height between 1 cm and 7 cm.

The ohmic resistance of the carrier structure also changes as a result of compression of the foamed material of the carrier structure. The inventors have discovered that if the force-resistance characteristic curve or the compression-resistance characteristic curve of the carrier structure is known, the currently acting compression force or the compression pressure can be determined. These properties or these characteristic curves can likewise be ascertained experimentally in advance, and are specific to the selected material and the selected geometry of the carrier structure. Via the current impinged via the second upper conductive layer and second lower conductive layer, and the associated picked-up voltage, the resistance of the carrier structure can be derived first, and hence the acting compression force can be determined.

This second upper conductive layer and the second lower conductive layer therefore serve to pick up a voltage dropped across the compressible carrier structure. In other words, a predefined current can be impinged onto the carrier structure via the two second conductive layers. The second conductive layers function, according to one or more example embodiments, as further measurement electrodes, which lie against the carrier structure. They are constituent parts of the voltage measurement apparatus according to one or more example embodiments, which, on the impinging of a defined current, acquire a voltage dropped across the carrier structure.

Furthermore, the statements above relating to the supporting structure and the first conductive layers apply to the carrier structure and to the second conductive layers.

In a further embodiment, the measurement system accordingly comprises a computing unit, which is designed to determine, under a patient load, a first prevailing compression force on the basis of the current impinged onto the supporting structure via the first upper conductive layer and the first lower conductive layer, and the voltage dropped across the supporting structure.

In further embodiments, the computing unit is further designed to determine, under a patient load, a second prevailing compression force on the basis of the current impinged onto the carrier structure via the second conductive layer, and the voltage dropped across the carrier structure.

The computing unit is designed to execute at least some of the steps of a method according to one or more example embodiments for measuring bio-electric signals, which method is described in greater detail later.

The computing unit can be embodied as an autonomous computing unit or as a part or component of the computing unit for signal analysis for a differential voltage measurement system according to one or more example embodiments, which system is described in greater detail below. The computing unit is consequently configured to determine, on the basis of the at least one value of the picked-up voltage and a characteristic curve held in a memory unit and retrievable for the supporting structure and/or the carrier structure, the prevailing first compression force, which is acting on the supporting structure, and a prevailing second compression force, which is acting on the carrier structure. The at least one characteristic curve can be held in the form of a look-up table, which associates defined voltage values with corresponding resistance values and/or directly with compression values and/or compression force values. In an alternative embodiment, in the memory unit is held a computing routine for the computing unit that uses the picked-up voltage value as an input value, and, on the basis thereof, with retrieval of a computing rule or a computing program, calculates a resistance value, a compression value and, therefrom or directly, a compression force value. The value of the imprinted current can also be input into the program routine as an input value.

In embodiments of the invention, the computing unit can be embodied as one or more central and/or distributed computing modules. Each of the computing modules can have one or more processors. A processor can be embodied as a central processing unit (CPU/GPU). Alternatively, the computing unit can be implemented as a local or cloud-based processing server. In addition, the computing unit can comprise one or more virtual machines.

The computing unit is further designed for data communication, in particular with the memory unit. The interface can be designed generally for data transfer between voltage measurement apparatus, comprising the first/second upper conductive layer and the first/second lower conductive layer, and/or the memory unit. The interface can thus be implemented in the form of one or more individual data interfaces, which can have a hardware and/or software interface, a data bus, for example a PCI bus, a USB interface, a FireWire interface, a ZigBee interface or a Bluetooth interface. The interface can also have an interface to a communication network, which communication network can have a local area network (LAN), for example an intranet or a wide area network (WAN). The one or more data interfaces can accordingly have a LAN interface or a wireless LAN interface (WLAN or Wi-Fi).

The result of the program routine is at least one value for the first compression force and one value for the second compression force that are currently acting on the measurement system, specifically on the sensor electrode and/or the supporting structure and/or the carrier structure.

In a further aspect, one or more example embodiments relates to a differential voltage measurement system for measuring bio-electric signals from a patient. The voltage measurement system has at least two signal measurement circuits, each corresponding to a wanted-signal path and each comprising a sensor electrode. At least one of the signal measurement circuits, preferably all comprised signal measurement circuits, comprise a measurement system according to one or more example embodiments, as described above.

The differential voltage measurement system according to one or more example embodiments acquires, as already mentioned in the introduction, bio-electric signals, for example from a human or animal patient. For this purpose, it has a number of measurement lines or wanted-signal paths. These connect, for example as individual cables, sensor electrodes, which are fastened to the patient for acquiring the signals, to further components of the voltage measurement system, i.e. in particular to electronics that are used to analyze and display the acquired bio-electric signals, in particular heartbeat signals.

The fundamental operating principle of differential voltage measurement systems is known to a person skilled in the art, and therefore is not explained in greater detail here. They can be embodied in particular as an electrocardiogram (EKG) device, an electroencephalogram (EEG) device, or an electromyogram (EMG) device. Particularly preferably, the differential voltage measurement system is integrated into a capacitive ECG mat comprising a multiplicity of sensor electrodes, onto which mat the patient simply lies for a measurement-signal acquisition. In further embodiments, the differential voltage measurement system is embodied differently.

In a preferred embodiment of the invention, the differential voltage measurement system comprises a control apparatus, which is designed to acquire from the computing unit via at least one interface the at least one first and/or at least one second prevailing compression force, and to compare each with at least one predefined first and/or second force threshold value respectively, and to produce, on the basis of the comparison, an output signal for an operator comprising a prompt to reposition the patient, and/or a control signal for parameterizing the signal measurement circuit. Specifically, the control signal for parameterizing is used to parameterize the analysis electronics of the signal measurement circuit.

In embodiments, the control apparatus comprises the computing unit of the at least one measurement system. The computing unit is then embodied as a submodule of the control apparatus. In other embodiments, these are embodied separately from each other. In either case, the computing unit and control apparatus are in contact via the interface for data transfer.

The control apparatus can additionally comprise at least one submodule for the analysis or further processing of signals of the analysis electronics of the differential voltage measurement system.

In addition, the above statements relating to the computing unit also apply to the control apparatus.

In an embodiment, the control apparatus is thus designed to perform, on the basis of the value of the prevailing first and/or second compression force, at least one step to check whether the patient must be placed in a new position relative to the measurement system.

For this purpose, the control apparatus can be designed in particular to perform a comparison of the first acquired compression force with at least one predefined first force threshold value, and, on the basis of the comparison, to produce an output signal for an operator comprising a prompt to reposition the patient. A first force threshold value can be specified in advance such that it equals the compression force at which the supporting structure is compressed exactly to the height of the frame structure. If this first force threshold value is reached, it is assumed according to one or more example embodiments that a good measurement contact or measurement distance between patient and sensor electrode is achieved. The patient is in an optimum position, and therefore in particular also highly sensitive signal measurements can be carried out. In embodiments, the first force threshold value can also be stated with a tolerance range of +/−5% around the value of the above compression force. If the value of the first acquired compression force lies within this tolerance range, the patient does not need to be repositioned. A further first force threshold value can be defined 20% to 30% lower than the above first force threshold value, for example at 75% or 80%. If the value of the first acquired compression force lies above the further first force threshold value and below the aforementioned first force threshold value, the measurement contact achieved with the present patient position may be sufficient for less sensitive signal measurements. The inventors have discovered that this value of the prevailing compression force is sufficient to compress the patient's clothing, for example, still sufficiently strongly for a capacitive signal measurement. Depending on a further signal indicating the nature of the signal measurement to be performed, the control apparatus can also be designed to determine whether or not the patient must be repositioned. If the result of the comparison is that the value of the first acquired compression force lies above the aforementioned first force threshold value or above its tolerance range, it is assumed according to one or more example embodiments that there is interference or an error, for example caused by an object or foreign body between patient and sensor electrode, and therefore the patient must in any case be repositioned, or the object must be removed. The control apparatus is accordingly designed to produce an output signal for an operator comprising a prompt to reposition the patient. In embodiments, this output signal can also correspond to a confirmation of the present patient position, namely when the value of the first prevailing compression force as described above is equal to the first aforementioned force threshold value, or, taking into account the signal measurement to be performed, deviates thereform within the stated bounds. Alternatively, the output signal can comprise the prompt for repositioning and/or an error message if the value of the first prevailing compression force lies above the first aforementioned force threshold value or below the further first force threshold value. If repositioning the patient or placing the patient in a new position cannot achieve an improved/sufficient/optimum measurement contact between sensor electrode and patient, the control apparatus is designed, in embodiments, to produce a control signal for re-parameterizing the analysis electronics of the signal measurement circuit in particular in order to adjust the sensitivity of the analysis electronics and still be able to carry out a signal measurement.

In addition, the control apparatus can be designed in particular to perform a comparison of the second acquired compression force with at least one predefined second force threshold value, and, on the basis of the comparison, to produce an output signal for an operator comprising a prompt to reposition the patient, or a control signal for parameterizing the signal measurement circuit. Specifically, the control signal is used to adjust the sensitivity of the analysis electronics of the signal measurement circuit.

In a preferred embodiment, the second force threshold value is designed to be like the first aforementioned force threshold value or slightly higher. If the value of the second acquired compression force lies above the second force threshold value, it is assumed that not only the supporting structure but also the carrier structure is compressed slightly by the patient's body. In such a way, variations in the total contact pressure, caused by patient movements related to breathing or heartbeat, have no effect, or only a slight effect, on the measurement contact between sensor electrode and patient. A signal measurement can be carried out reliably. Repositioning is unnecessary.

If the value of the second acquired compression force lies below the second predefined force threshold value, the above-described movement compensation is not guaranteed, and the patient must be repositioned.

In embodiments, the output signal can thus correspond to a confirmation of the present patient position, namely when the value of the second prevailing compression force as described above is greater than or equal to the second aforementioned force threshold value. Alternatively, the output signal can comprise the prompt for repositioning and/or an error message if the value of the second prevailing compression force lies below the second force threshold value. If repositioning the patient or placing the patient in a new position cannot bring the value of the second acquired compression force above the second force threshold value, the control apparatus is designed again here, in embodiments, to produce a control signal for re-parameterizing the analysis electronics of the signal measurement circuit in particular in order to adjust the sensitivity of the analysis electronics and still be able to carry out a signal measurement.

A method according to one or more example embodiments for measuring bio-electric signals from a patient via a differential voltage measurement system according to one or more example embodiments comprising at least one measurement system according to one or more example embodiments comprises, in a further aspect, a multiplicity of steps. The method is at least partially a computer-implemented method.

In a first optional step, positioning of the patient on the differential voltage measurement system is performed. The patient is consequently brought initially into spatial proximity with the at least one measurement system, in particular with its measurement electrode. Alternatively, the patient is already in place against or on the at least one measurement system.

A further step is directed at impinging a current onto the compressible conductive supporting structure via the first upper conductive layer and the first lower conductive layer.

A further step comprises acquiring via the first conductive layers a voltage dropped across the supporting structure.

In a further step, determining a first prevailing compression force is performed via a computing unit.

In a further step, comparing the first prevailing compression force with at least one first force threshold value is performed via the control apparatus.

In a further step, producing a first output signal for an operator comprising a prompt to reposition the patient and/or a first control signal for parameterizing the signal measurement circuit is performed via the control apparatus.

A preferred embodiment of the method according to the invention further comprises the following steps:
impinging a current onto the compressible conductive carrier structure via the second upper conductive layer and the second lower conductive layer;
acquiring via the second conductive layers a voltage dropped across the carrier structure;
determining a second prevailing compression force via the computing unit;
comparing the second prevailing compression force with at least one second force threshold value via the control apparatus; and
producing via the control apparatus a second output signal for an operator comprising a prompt to reposition the patient and/or a second control signal for parameterizing the signal measurement circuit.

In a particularly preferred embodiment of the method, the method steps are executed repeatedly. In this case, the patient is repositioned or placed in a new position preferably after each repetition loop. This method is carried out particularly preferably during a signal measurement, and hence is used to monitor the measurement-contact quality. In embodiments, a break criterion for the method repetition can be met if the value of the first and/or second acquired prevailing compression force reaches a value that corresponds to a good/sufficient measurement-contact quality.

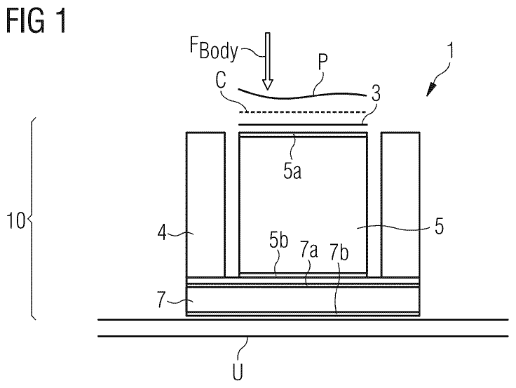

FIG. 1 shows a view of a measurement system 1 in an exemplary embodiment of the invention in a first loading state. The measurement system 1 shown is used to measure bio-electric signals from a patient P, who is positioned on the measurement system 1 in this embodiment. A weight FBody therefore acts on the measurement system 1. This comprises a sensor electrode in the form of a flat electrode 3. This is covered by, for example, a textile layer C, the clothing of the patient P. Also comprised is a mechanical mounting 10 for the sensor electrode 3. This is designed to be compressible, i.e. it can be compressed, at least partially, so at least in portions. The mechanical mounting 10 comprises a frame structure 4 and a supporting structure 5. The supporting structure 5 is arranged beneath the sensor electrode, and has a base area equal to at least the base area of the sensor electrode 3, so that the sensor electrode 3 rests entirely on the supporting structure 5. The more exactly the two base areas match each other the better. The supporting structure can also have a larger base area than the sensor electrode 3, however. The supporting structure carries the sensor electrode 3. The mechanical mounting 10 is attached to a substrate U of the measurement system 1, and supports the sensor electrode 3 against the substrate U. The substrate is formed, for example, by the underside/supporting surface of a capacitive patient mat for measuring ECG signals. The frame structure 4 at least partially surrounds the supporting structure 5. In this embodiment, the frame structure 4 is provided on two sides of the supporting structure 5. In alternative embodiments, the frame structure 5 can be a closed structure around the supporting structure 5.

The supporting structure 5 is designed to be higher than the frame structure 4, or protrudes, in the unloaded state, beyond this. Thus, in the unloaded state, it extends further from the substrate U toward the patient P than the frame structure 4.

In this embodiment, the supporting structure 5 is designed to be compressible. More precisely, the supporting structure 5 is formed from a compressible foamed material. In addition, the supporting structure is conductive, i.e. formed from a conductive material. Above and beneath the supporting structure 5 is arranged respectively a first upper conductive layer and a first lower conductive layer 5a, 5b. The first conductive layers 5a, 5b act as measurement electrodes, via which a current is impinged onto the supporting structure 5, and a voltage dropped thereacross can be picked up. In the present case, the supporting structure 5 is made from a PU foam with a 45% carbon admixture. The first conductive layers 5a, 5b are formed by a PU plastic with again a 45% carbon admixture. The PU plastic is designed to be highly conductive.

The frame structure 4 can likewise be made from a compressible material, in particular a foamed material (enhances patient comfort). It can, however, also consist of an incompressible material, for example a plastic or wood. In each case, the supporting structure has a lower hardness than the frame structure 4. The supporting structure 5 consequently has a higher compressibility than the frame structure 4. In each case, the frame structure 4 must be selected to be firm enough to be able to hold the supporting structure 5 substantially at the height of the frame structure 4 continuously and over a prolonged period.

The supporting structure 5 is then compacted substantially to the height of the frame structure 4 by a force component of the weight FBody of the patient P. The excess/remaining portion of the weight is transferred to the frame structure 4, which is less compressible or incompressible. The force component acting on the sensor electrode 3 or the supporting structure 5 causes a counterforce toward the patient P, which counterforce is produced by the supporting structure 5 and is equal in magnitude to the force component. This counterforce then remains constant even during patient movement, for instance through a shift in weight, or related to heartbeat and/or breathing, because the frame structure 4 predetermines a maximum compression of the supporting structure 5. According to one or more example embodiments, a variation in the acting weight FBody affects only the frame structure 4 or the underlying carrier structure 7, but no longer affects the supporting structure 5.

The measurement system 1 shown thus comprises a carrier structure 7 as a further constituent part of the mechanical mounting 10. This is arranged beneath supporting structure 5 and frame structure 4, and above the substrate U. In this embodiment, the carrier structure 7 covers the entire surface of the substrate over the base area of the measurement system 1. In this embodiment, the carrier structure 7 is likewise made of a compressible PU foamed material, and serves to achieve a comfortable support for the patient P despite the hard frame structure 4, or in other words to absorb the excess force component of the weight FBody. For this purpose, the carrier structure 7 is designed also to be softer than the frame structure 4 but harder than the supporting structure 5. In the present case, the carrier structure 7 is designed to be 25% harder than the supporting structure 5. A portion of the excess weight FBody acting on the frame structure 4 can thus be introduced into the carrier structure 7, compressing it and causing the frame structure 4 and supporting structure 5, together with sensor electrode 3, to yield jointly according to the weight, with a stable measurement contact between patient and sensor electrode 3. Thus the carrier structure 7 would also be compressed by a varying amount during a patient movement. The counterforce acting on the sensor electrode would be kept substantially constant, however. The carrier structure 7 also allows equalization of different height levels of the patient surface, in particular in configurations in which the measurement system 1 is integrated multiple times in a capacitive patient mat, for example.

In addition, the carrier structure 7 is also conductive, i.e. formed from a conductive material. Above and beneath the carrier structure 7 is arranged respectively a second upper conductive layer and a second lower conductive layer 7a, 7b. The second conductive layers 7a, 7b likewise act as measurement electrodes, via which a current is impinged onto the carrier structure 7, and a voltage dropped thereacross can be picked up. In the present case, the carrier structure 7 is formed with a 35% carbon admixture. The second conductive layers 7a, 7b are formed by a PU plastic with again a 45% carbon admixture. The PU plastic is designed to be highly conductive.

The base area of the supporting structure 5 is square in this case, with dimensions 3 cm×3 cm, corresponding to the base area of the sensor electrode 3. The height of the supporting structure 5 is 8 cm.

In general, the choice of the supporting-structure foamed material and the heights of the supporting structure 5 and frame structure 4 are selected such that 10 N to 50 N force components are sufficient to compress the supporting structure 5 of a measurement system 1 to the height of the frame structure 4. This ensures, even for light patients P, compression of the supporting structure 5 that is sufficient according to one or more example embodiments, and ensures a contact force of corresponding magnitude on the sensor electrode 3 that allows stable signal acquisition.

The frame structure also has a depth of 3 cm corresponding to the sensor electrode 3 or the supporting structure 5, and a thickness of 1.5 cm. In embodiments, at least the frame structures of directly adjacent measurement systems 1 can be formed as a single piece or joined to one another. This advantageously enlarges the supporting surface for the patient, thereby increasing patient comfort. The frame structure 4 here consists of a substantially incompressible plastic. The carrier structure 7 has a height of 7 cm.

The individual structures/layers of the mechanical mounting 10 can be welded or adhesively bonded to one another. Alternatively, at least the supporting structure 5 can be inserted, and hence fixed, between each of the frame structures 4 or in a frame structure 4.

In the present case, the conductive first and second layers 5a, 5b, 7a, 7b are welded to the supporting structure 5 and the carrier structure respectively, in order to minimize the resistance at the interfaces between conductive layer and supporting structure or carrier structure 5, 7 respectively.

In the present case, the conductive first and second layers 5a, 5b, 7a, 7b are formed from a highly conductive plastic having a surface resistance of a few kilohms, for example 3 kilohms. The supporting structure 5 and the carrier structure 7 have surface resistances between 100 kilohms and 1000 kilohms, for example 200 kilohms, 500 kilohms or 700 kilohms. In the present case, the conductive first and second layers 5a, 5b, 7a, 7b have a thickness of 35 μm.

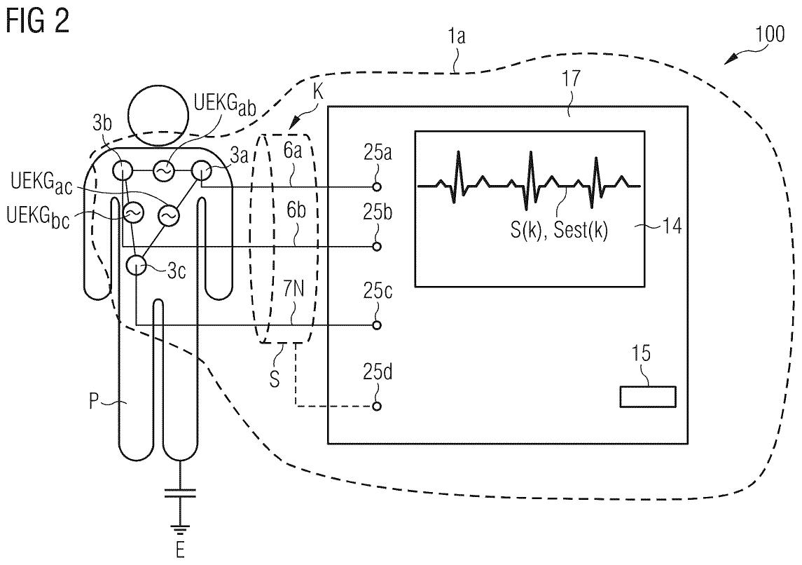

FIG. 2 shows a view of a differential voltage measurement system 100 in the form of an ECG facility, which is arranged on a patient P. The general operating principle of an ECG facility is described with reference to FIG. 2. The voltage measurement system 100 comprises an ECG device 17 together with its electrical connectors, and sensor electrodes 3a, 3b, 3c connected thereto via cables K in order to measure ECG signals S(k) at the patient P. At least one, preferably all the sensor electrodes 3a, 3b, 3c, can be part of a measurement system 1 according to one or more example embodiments as described in the further figures.

In order to measure the ECG signals S(k), at least a first electrode 3a and a second electrode 3b are needed, which are fastened against, on or under the patient P. The electrodes 3a, 3b are connected by signal measurement cables K to the ECG device 17 via connectors 25a, 25b, usually plug-in connections. The first electrode 3a and the second electrode 3b including the signal measurement cables K form part of a signal acquisition unit, which can be used to acquire the ECG signals S(k). The signal acquisition unit further comprises the analysis electronics for further processing of measurement signals from the individual measurement systems.

A third electrode 3c serves as a reference electrode for providing potential equalization between the patient P and the ECG device 17. It is the convention to fasten this third electrode 3c to the right leg of the patient (“right-leg drive” or “RLD”). It can, however, also be positioned at another location. Furthermore, via further connectors (not shown here) on the ECG device 17, a multiplicity of further contacts for further derivations (measurements of potential) can be fastened to the patient P and used to form suitable signals.

Between the individual electrodes 3a, 3b, 3c are formed the voltage potentials UEKGab, UEKGbc and UEKGac used for measuring the ECG signals S(k).

The directly measured ECG signals S(k) are displayed on a user interface 14 of the ECG device 27.

During the ECG measurement, the patient P is coupled at least capacitively to the ground potential E (depicted by a coupling on the right leg).

The signal measurement cables K that lead from the first sensor electrode 3a and the second sensor electrode 3b to the ECG device 17 are part of the wanted-signal paths 6a, 6b or of the corresponding sensor lines. The signal measurement cable K that leads from the electrode 3c to the ECG device 17 here corresponds to part of a third wanted-signal path 7N. The third wanted-signal path 7N can serve in particular to transmit interference signals that have been coupled in via the patient P and the electrodes.

The cables K have a shield S, which is depicted here schematically as a dashed cylinder surrounding all the wanted-signal paths 6a, 6b, 7N. The shield does not have to surround all the cables K jointly, however; instead, the cables K can also be shielded separately. The connectors 25a, 25b, 25c, however, preferably each have an integral pin for the shield S. These pins are then taken jointly to a common shield connector 25d. The shield S is here embodied, for example, as a metal foil, which surrounds the conductor of each cable K but is insulated from the conductor.

In addition, the ECG device 17 can have an external interface 15, for instance in order to provide a connection for a printer, a storage means and/or even a network. The ECG device 17 has signal measurement circuits 40 (see e.g. FIG. 6) according to exemplary embodiments of the invention, which signal measurement circuits are assigned to the respective connectors 25a, 25b. The signal measurement circuits 40 are each again connected to ground E via a ground switch 31.

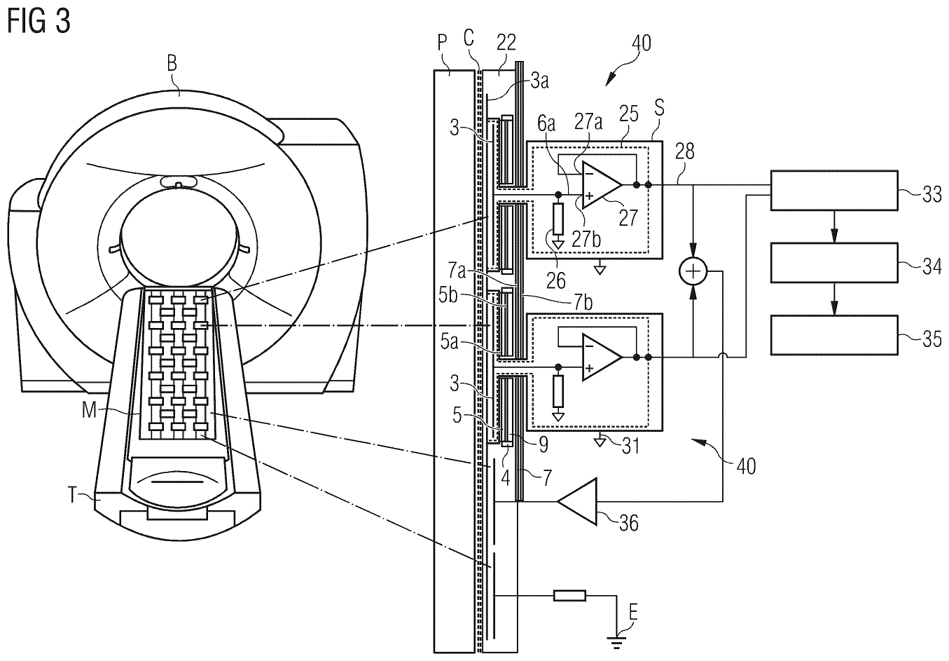

FIG. 3 shows a view of a differential voltage measurement system 100 comprising two signal measurement circuits 40 according to one or more example embodiments. The two signal measurement circuits are identical in design, and therefore, for the sake of clarity, corresponding components have mostly been given a reference sign just once.

The arrangement of a single sensor 3 or a single sensor electrode 3 of the signal measurement circuit 40 is illustrated here in the form of a capacitive ECG measurement circuit. Patient P and sensor electrode 3 are in spatial proximity to one another. Stated more precisely, patient P is here lying on a patient table T of an imaging modality B in the form of a computed tomography facility. A capacitive patient mat M, on which the patient P is positioned, is arranged on the patient table. The patient mat M comprises a multiplicity of signal measurement circuits 40 according to one or more example embodiments. The mat M can alternatively be configured as an electrode pad which can be arranged in particular in a backrest of an examination or treatment chair. Two of the signal measurement circuits 40 are described in greater detail below. Patient P can be provided, for example, with fabric clothing C. Optionally lying thereupon is a cover 22 which is transparent to X-rays. The sensor electrode 3 is not in direct electrical contact with the patient P, but is instead electrically insulated from the patient P at least by a sensor covering 3a. However, the sensor covering 3a does not impair a capacitive coupling-in of an ECG signal. The sensor electrode 3, a sensor line 6a running from the sensor electrode 3 to an operational amplifier 27, and the measurement circuit 40 comprising the operational amplifier 27 are surrounded by what is known as an active guard 25 and preferably a shield S. The operational amplifier 27 is embodied as what is known as a voltage follower. This means that the negative input 27a of the operational amplifier 27 is coupled to the output 28 of the operational amplifier 27. A high virtual input impedance is thereby achieved for the operational amplifier 27 at the positive input 27b. This means that because of the voltage adaptation between the output 28 and the positive input 27b, barely any current flows between the sensor 3 and the active guard 25. Furthermore, the positive input 27b of the operational amplifier 27 is maintained at an electrical bias voltage with the aid of a resistor 26 connected to the measurement device ground (also called “measurement ground”). Thereby, the positive input can be set to a desired measurement potential. DC components are suppressed in this way. This is desirable since the intention is for the sensor electrode 3 to couple primarily capacitively, and to avoid a varying potential.

The signal measurement circuits 40 shown each comprise a measurement system 1, for example corresponding to further figures, each comprising a mechanical mounting 10 having a supporting structure 5, a frame structure 4 and a carrier structure 7. Supporting structure 5 and carrier structure 7 are each covered by respective first and second conductive layers 5a, 5a, 7a, 7b, as already described with reference to FIG. 1. The active guard 25 and shield S each enclose the sensor electrode 3 in order to shield it effectively. The active guard and shield S further enclose the sensor line 6a, and together with it, penetrate the supporting, intermediate and carrier structures 5, 9, 7 on the way to the operational amplifier 27. The carrier structure 7 is here formed as a single piece and acts equivalently for both signal measurement circuits 40. Alternative arrangements of the sensor line 6a are obviously also conceivable.

A further electrode is also provided in the patient mat M shown here for at least capacitive coupling of the patient to the earth potential E.

A further electrode or the associated measurement circuit 36 functions in the patient mat M as a reference electrode, for instance as what is known as a driven neutral electrode (DNE).

The differential voltage measurement system 100 further comprises a switching apparatus in the form of a switch matrix 33. In the case of a multiplicity of sensor electrodes 3, it serves to select which of the sensor electrodes are used for further signal processing.

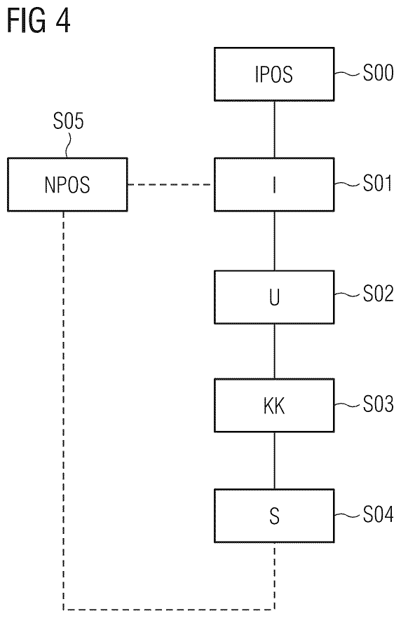

The differential voltage measurement system 100 further comprises a signal processing apparatus in the form of a signal processing box 34. This is designed to perform pre-processing of the acquired measurement signals in order to remove interference components. The signal processing apparatus 34 can be designed to execute standard processing using frequency-based filters such as band-pass or band-stop filters, but also advanced interference suppression such as, for example, in German patent application DE 102019203627A. In the present case, the signal processing apparatus 34 also comprises, or is embodied as, the control apparatus of the differential voltage measurement system. In this case, the control apparatus comprises as submodules the computing units of the individual measurement systems 1. The signal processing apparatus 34 is therefore designed to perform steps of the method according to one or more example embodiments, as described in greater detail with reference to FIG. 4. In particular, the signal processing apparatus 34 is designed to determine at least one value for a first and/or second currently acting compression force KK1, KK2. It is further configured to perform a comparison between the value for a first and/or second currently acting compression force KK1, KK2 with at least one predefined first and/or second force threshold value S1, S2, and, on the basis of the comparison, to produce an output signal for an operator comprising a prompt to reposition the patient, and/or a control signal for parameterizing the signal measurement circuit.

In addition, the differential voltage measurement system 100 comprises a trigger apparatus 35. This is designed to perform a method for identifying a heartbeat of the patient P and/or the heart rhythm, in order to generate therefrom control signals comprising trigger or start-time information for a medical imaging facility. On the basis of the control signals from the trigger apparatus 35, the imaging facility calculates the time points for an image data acquisition.

The embodiments in FIGS. 1 to 3 relate, in principle, to arrangements in which a patient P is in a lying position and his weight FBody is acting upon at least one measurement system 1 according to one or more example embodiments. Example embodiments are not restricted thereto, however. A measurement system 1 according to one or more example embodiments can advantageously also be used for sitting positions of the patient P or suchlike.

FIG. 4 shows a schematic diagram of a method according to one or more example embodiments. The partially computer-implemented method for measuring bio-electric signals (S(k)) from a patient P via a differential voltage measurement system 100 according to one or more example embodiments comprising at least one measurement system 11 according to one or more example embodiments comprises the following steps:

Step S00 comprises an initial positioning of the patient P on the differential voltage measurement system 100 according to an initial position IPOS. Alternatively, step S00 of the initial positioning can also precede the method according to one or more example embodiments. In this case, the method according to one or more example embodiments starts with step S01. In either case, however, initial positioning of the patient P relative to the differential voltage measurement system 100, or vice versa, takes place.

Step S01 relates to impinging a current I. Specifically, step S01 comprises impinging a current I1 onto the compressible conductive supporting structure 5 via the first upper conductive layer and the first lower conductive layer 5a, 5b. A predefined current I1 is thus introduced into the supporting structure 5 via the voltage measurement apparatus comprising the first conductive layers 5a, 5b. As a result of the initial positioning of the patient P, the supporting structure 5 is in a compressed state, wherein the resistance of the supporting structure 5 decreases with the degree of compression.

Optionally, step S01 also comprises impinging a current I2 onto the compressible conductive carrier structure 7 via the second upper conductive layer and the second lower conductive layer 7a, 7b. As a result of the initial positioning of the patient P, the carrier structure 7 may also be in a compressed state, and the carrier structure 7 may thereby have a resistance that is lower compared with the unloaded state.

A further step S02 comprises acquiring a voltage U. Specifically, a voltage U1 dropped across the supporting structure 5 is acquired via the first conductive layers 5a, 5b of the voltage measurement apparatus.

Optionally, step S02 also comprises acquiring via the second conductive layers 7a, 7b of the voltage measurement apparatus a voltage U2 dropped across the carrier structure 7.

In step S03, at least one compression force KK is determined. A first prevailing compression force KK1 is determined via the computing unit. In this process, initially the currently effective resistance of the supporting structure 5 is determined, and a value for a first compression force KK1 acting on the supporting structure is ascertained, for instance on the basis of characteristic curves or lookup tables.

Optionally, step S03 comprises determining a second prevailing compression force KK2 via the computing unit. In this process, initially the currently effective resistance of the carrier structure 7 is determined, and a value for a second compression force KK2 acting on the carrier structure 7 is ascertained, for instance on the basis of characteristic curves or lookup tables.

In a further step S04, a prevailing compression force KK is compared with at least one force threshold value S via the control apparatus. Specifically, the first prevailing compression force KK1 is compared with at least one first force threshold value S1.

Optionally in step S04, the second prevailing compression force KK2 is compared with at least one second force threshold value S2.

Step S04 further comprises producing a first output signal and optionally a second output signal for an operator comprising a prompt to reposition the patient P and/or a first and second control signal respectively for parameterizing the signal measurement circuit 40. The output signal and the control signal are produced here according to, or on the basis of, the comparison result.

A first and/or second output signal can comprise, according to the comparison result, indicator information that the patient is well positioned and sufficiently good measurement contact is made. Alternatively, the output signal can comprise indicator information that comprises a prompt to reposition the patient. Alternatively, the output signal can comprise an error message. The output signal can be output via the user interface 14, for example.

A first/second control signal for parameterizing the signal measurement circuit 40 comprises control information for adjusting the sensitivity of the analysis electronics of at least one of the signal measurement circuits in order to be able to carry out a measurement of bio-electric signals even when the patient positioning is not optimum, in particular when this cannot be optimized.

The method according to one or more example embodiments can be carried out repeatedly in particular accompanying a signal measurement, in order to monitor the quality of the measurement contact and hence the quality of the bio-electric signals S(k). Optionally here, a step S05 can perform a repositioning of the patient P into a new position or placement NPOS in order to improve the measurement contact.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Even if not explicitly stated, individual exemplary embodiments, or individual sub-aspects or features of these exemplary embodiments, can be combined with, or substituted for, one another, if this is practical and within the meaning of the invention, without departing from the present invention. Without being stated explicitly, advantages of the invention that are described apply to other exemplary embodiments, where transferable.

1. A measurement system for measuring bio-electric signals from a patient, comprising:
a sensor electrode; and
a mechanical mounting for the sensor electrode, the mechanical mounting being compressible at least partially by a weight of the patient, the mechanical mounting including a frame structure and a compressible supporting structure,
wherein
the mechanical mounting is attachable to a substrate of the measurement system to support the sensor electrode against the substrate,
the compressible supporting structure is beneath the sensor electrode,
the frame structure at least partially surrounds the compressible supporting structure, and
in an unloaded state, the compressible supporting structure protrudes beyond the frame structure,
wherein the compressible supporting structure is conductive, the compressible supporting structure is covered in a direction of the sensor electrode and in a direction of the substrate by a first upper conductive layer and a first lower conductive layer, respectively, the first upper conductive layer and the first lower conductive layer form first conductive layers, and the first conductive layers are configured to generate a voltage drop across the compressible supporting structure.

2. The measurement system as claimed in claim 1, wherein the compressible supporting structure has a lower hardness than the frame structure.

3. The measurement system of claim 1, wherein the compressible supporting structure comprises a conductive foamed material.

4. The measurement system of claim 1, wherein the first conductive layers comprise a conductive plastic.

5. The measurement system of claim 1, wherein the compressible supporting structure, in an unloaded state, has an ohmic resistance between 100 kilohms and 1000 kilohms.

6. The measurement system of claim 1, further comprising:
a computing unit configured to determine, under a patient load, a first prevailing compression force based on a current impinged onto the compressible supporting structure via the first conductive layers, and the voltage drop across the compressible supporting structure.

7. The measurement system of claim 1, wherein the first conductive layers have a height in a range of 20 μm to 50 μm.

8. The measurement system of claim 2, wherein at least one of the compressible supporting structure or the first conductive layers comprise a carbon-enriched material including at least one of polyurethane or polyethylene terephthalate, wherein a carbon content is between 20 to 50 percent by volume.

9. The measurement system of claim 1, wherein the mechanical mounting further comprises:
a carrier structure, the carrier structure being compressible by the weight of the patient and extending beneath the compressible supporting structure and the frame structure, the carrier structure being conductive and covered in the direction of the sensor electrode and in the direction of the substrate by second conductive layers, respectively, to acquire a voltage drop across the carrier structure.

10. The measurement system of claim 9, wherein the computing unit is configured to determine, under a patient load, a second prevailing compression force on the basis of a current impinged onto the carrier structure via the second conductive layers, and the voltage drop across the carrier structure.

11. The measurement system of claim 9, wherein the compressible supporting structure has a lower hardness than the carrier structure.

12. A signal measurement circuit for a differential voltage measurement system, comprising:
the measurement system of claim 1;
a measurement amplifier circuit; and
a sensor line between the measurement amplifier circuit and the sensor electrode.

13. A differential voltage measurement system comprising:
at least two signal measurement circuits, each of the at least two signal measurement circuits corresponding to a wanted-signal path, wherein at least one of the two signal measurement circuits comprises the measurement system of claim 1.

14. The differential voltage measurement system of claim 13, further comprising:
a control apparatus configured to acquire from a computing unit via at least one interface unit at least one of at least one first prevailing compression force or at least one second prevailing compression force, the control apparatus configured to
compare each of the at least one of at least one first prevailing compression force or at least one second prevailing compression force with at least one of at least one predefined first threshold value or at least one second force threshold value, and
produce, based on the comparison, an output signal for an operator, the output signal comprising at least one of a prompt to reposition the patient or a control signal for parameterizing at least one of the at least two signal measurement circuits.

15. A method for measuring the bio-electric signals via the differential voltage measurement system of claim 13, the method comprising:
positioning the patient on the differential voltage measurement system;
impinging a current onto the compressible supporting structure via the first conductive layers;
acquiring via the first conductive layers the voltage drop across the compressible supporting structure;
determining a first prevailing compression force via a computing unit;
comparing the first prevailing compression force with at least one first force threshold value via a control apparatus; and
producing via the control apparatus a first output signal for an operator, the first output signal comprising at least one of a prompt to reposition the patient or a first control signal for parameterizing at least one of the at least two signal measurement circuits.

16. The method for measuring bio-electric signals from a patient as claimed in claim 15 wherein the mechanical mounting further includes a carrier structure, the carrier structure being compressible by the weight of the patient and extending beneath the compressible supporting structure and the frame structure, the carrier structure being conductive and covered in the direction of the sensor electrode and in the direction of the substrate by second conductive layers, respectively, to acquire a voltage drop across the carrier structure, the method further comprising:
impinging a current onto the carrier structure via the second conductive layers;
acquiring via the second conductive layers the voltage drop across the carrier structure;
determining a second prevailing compression force via the computing unit;
comparing the second prevailing compression force with at least one second force threshold value via the control apparatus; and
producing via the control apparatus a second output signal for an operator, the second output signal comprising a prompt to reposition the patient or a second control signal for parameterizing at least one of the at least two signal measurement circuits.

17. The method of claim 15, which is executed repeatedly during the measurement of the bio-electric signals.

18. The measurement system of claim 6, wherein the first conductive layers have a height in a range of 20 μm to 50 μm.

19. The measurement system of claim 18, wherein at least one of the compressible supporting structure or the first conductive layers comprise a carbon-enriched material including at least one of polyurethane or polyethylene terephthalate, wherein a carbon content is between 20 to 50 percent by volume.

20. The measurement system of claim 19, wherein the mechanical mounting further comprises:
a carrier structure, the carrier structure being compressible by the weight of the patient and extending beneath the compressible supporting structure and the frame structure, the carrier structure being conductive and covered in the direction of the sensor electrode and in the direction of the substrate by second conductive layers, respectively, to acquire a voltage drop across the carrier structure.
